(19) United States
(12) Patent Application Publication
(10) Pub. No.: US 2024/0310342 A1
(43) Pub. Date: Sep. 19, 2024

(54) GAS DETECTION SYSTEM AND GAS DETECTION METHOD

(71) Applicant: KABUSHIKI KAISHA TOSHIBA, Tokyo (JP)

(72) Inventors: Dean KOS, Ota Tokyo (JP); Yasushi Shinjo, Kawasaki Kanagawa (JP); Isao Takasu, Setagaya Tokyo (JP); Kohei Nakayama, Kawasaki Kanagawa (JP); Akiko Hirao, Kawasaki Kanagawa (JP)

(73) Assignee: KABUSHIKI KAISHA TOSHIBA, Tokyo (JP)

(21) Appl. No.: 18/305,440

(22) Filed: Apr. 24, 2023

(30) Foreign Application Priority Data
Mar. 15, 2023 (JP) 2023-041090

(51) Int. Cl. G01N 33/00; G01N 27/407

(52) U.S. Cl. CPC G01N 33/0011; G01N 27/407; G01N 21/31; G01N 2201/12

(57) ABSTRACT

A gas detection system includes: a gas detector that detects a first analyte in a first sample gas to generate a first detection signal; a signal generator that generates a first reference signal corresponding to the first analyte; a signal processor that performs first signal processing using the first detection signal and the first reference signal, to thereby generate a first processed signal; and a gas controller that varies the concentration of the first analyte in the first sample gas with time based on the first reference signal so as to make the first detection signal synchronize with the first reference signal.

### CROSS-REFERENCE TO RELATED APPLICATIONS

This application is based upon and claims the benefit of priority from Japanese Patent Applications No. 2023-41090, filed on Mar. 15, 2023, the entire contents of which are incorporated herein by reference.

### FIELD

Embodiments relate to a gas detection system and a gas detection method.

### BACKGROUND

In recent years, known examples of gas detection systems can detect an analyte in a measuring object gas. Some gas detection system decreases its detection sensitivity due to noise in a detection signal.

### BRIEF DESCRIPTION OF THE DRAWINGS

FIG. 1 is a schematic diagram illustrating a configuration example of a gas detection system in a first embodiment;

FIG. 2A is a diagram illustrating a configuration example of a lock-in amplifier;

FIG. 2B is a diagram for explaining the principle of the lock-in amplifier;

FIG. 3 is a schematic view illustrating another example of a conventional gas detection system;

FIG. 4 is a view illustrating an example of a comparison result between an SN ratio of a processed signal PS of the gas detection system in the first embodiment and an SN ratio of a detection signal of the gas detection system in a comparative embodiment illustrated in FIG. 3;

FIG. 5 is a schematic diagram illustrating a configuration example of a gas detection system in a second embodiment;

FIG. 6 is a schematic diagram illustrating a configuration example of a gas detection system in a third embodiment;

FIG. 7 is a schematic diagram illustrating a configuration example of a gas detection system in a fourth embodiment; and

FIG. 8 is a schematic diagram illustrating a configuration example of a gas detection system in a fifth embodiment.

### DETAILED DESCRIPTION

The gas detection system in the embodiment includes: a gas detector that detects a first analyte in a first sample gas to generate a first detection signal; a signal generator that generates a first reference signal corresponding to the first analyte; a signal processor that performs first signal processing using the first detection signal and the first reference signal, to thereby generate a first processed signal; and a gas controller that varies the concentration of the first analyte in the first sample gas with time based on the first reference signal so as to make the first detection signal synchronize with the first reference signal.

Hereinafter, there will be explained embodiments with reference to the drawings. In each of the embodiments to be described below, substantially the same components are denoted by the same reference numerals and symbols, and a description thereof may be partly omitted. The drawings are schematic, and a relation between thickness and planar dimension, a thickness ratio among parts, and so on may be different from actual ones.

In this specification, “connecting” includes not only directly connecting but also indirect connecting, unless otherwise specified.

### First Embodiment

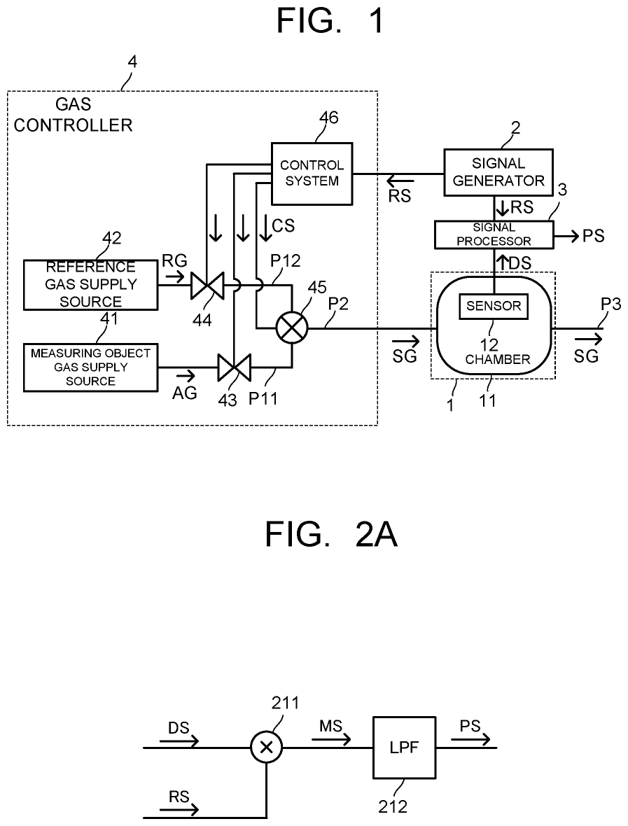

FIG. 1 is a schematic diagram illustrating a configuration example of a gas detection system in a first embodiment. The gas detection system illustrated in FIG. 1 includes a gas detector 1, a signal generator 2, a signal processor 3, and a gas controller 4. The gas detection system may include a control device that controls the gas detector 1, the signal generator 2, the signal processor 3, and the gas controller 4, and a power supply device that supplies power to the gas detector 1, the signal generator 2, the signal processor 3, and the gas controller 4.

The gas detector 1 can detect an analyte in a sample gas SG to generate a detection signal DS. Examples of the gas detector 1 include a chamber 11 and a sensor 12, as illustrated in FIG. 1.

The chamber 11 has a space allowing the sample gas SG to pass therethrough. The chamber 11 is connected to an inlet for supplying the sample gas SG to the chamber 11 and an outlet for discharging the sample gas SG from the chamber 11. The inlet is connected to a pipe P2. The outlet is connected to a pipe P3. The introduction and discharge of the sample gas SG may be controlled by a pump, which is provided in the middle of the pipe P2 and the pipe P3, for example.

The volume of the chamber 11 is preferably small, which is preferably, for example, 1 cm³ or less. A decrease of the volume of the chamber 11 can improve the response speed of the sensor 12.

The sensor 12 is provided in the chamber 11. The sensor 12 is a gas sensor capable of detecting an analyte in the sample gas SG flowing through the chamber 11 to generate an electrical signal. The voltage of the electrical signal varies according to the type and concentration of the analyte. Examples of the sensor 12 include a concentration sensor, a semiconductor gas sensor, and an electrochemical gas sensor.

The sensor 12 may include a field-effect transistor and an organic probe provided on a channel formation region of the field-effect transistor. The organic probe uses an organic compound that selectively binds to the analyte. When a current is applied between a source and a drain of the field-effect transistor with a voltage applied to a gate of the field-effect transistor and the analyte is captured by the organic probe, the voltage of the gate of the field-effect transistor varies. Measurement of this electrical variation can detect the analyte.

The ratio of a detection area of the sensor 12 to a flow path surface area, defined by the product of a flow path length and a flow path width of the chamber 11, is preferably large, which is preferably, for example, 0.5 or more. An increase of the ratio of the detection area to the flow path surface area, can improve the detection sensitivity of the sensor 12.

The signal generator 2 can generate a reference signal RS corresponding to the analyte. The signal generator 2 includes a signal oscillator that oscillates the reference signal RS, for example. Examples of the reference signal RS include a periodic signal such as a sine wave, a square wave, and a triangular wave. The waveform of the reference signal RS can be set in advance, for example, by the signal oscillator according to the type of analyte.

The signal processor 3 can generate a processed signal PS by performing signal processing using the detection signal DS input from the gas detector 1 and the reference signal RS input from the signal generator 2. Examples of the signal processing include noise removal. The signal processor 3 includes a signal processing device, for example. The signal processor 3 includes a lock-in amplifier, for example.

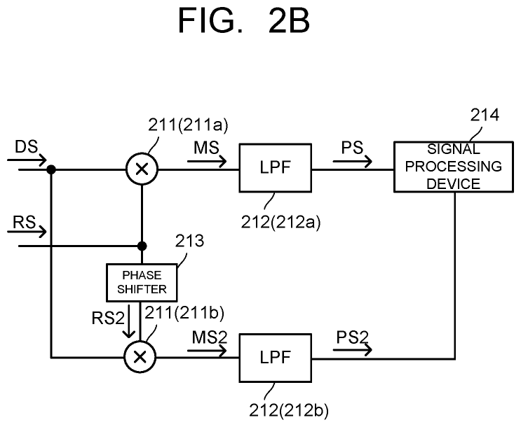

FIG. 2A is a diagram illustrating a configuration example of the lock-in amplifier. The lock-in amplifier illustrated in FIG. 2A includes a multiplying circuit 211 and a low-pass filter (LPF) 212. The lock-in amplifier illustrated in FIG. 2A can generate the direct-current processed signal PS in a manner that the multiplying circuit 211 performs multiplication processing using the detection signal DS input from the gas detector 1 and the reference signal RS input from the signal generator 2 and the low-pass filter 212 cuts specific frequency components from an obtained multiplication signal MS.

The lock-in amplifier can generate the multiplication signal MS and shift the peak based on the analyte in a signal waveform Fourier transformed by the signal processing device and extract it as a direct-current component. This allows the component based on the analyte to be extracted using the low-pass filter with a simple circuit configuration, without using a filter with a complex circuit configuration such as a band-pass filter. Therefore, the detection sensitivity of the analyte can be improved.

FIG. 2B is a diagram for explaining the principle of the lock-in amplifier. FIG. 2B illustrates a plurality of multiplying circuits 211 (211a, 211b), a plurality of low-pass filters 212 (212a, 212b), a phase shifter 213, and a signal processing device 214. As illustrated in FIG. 2B, after the reference signal RS is divided, the phase shifter 213 is used to generate a reference signal RS2 in which the phase of the reference signal RS is advanced by 90 degrees. Then, the multiplying circuit 211 (multiplying circuit 211b) performs multiplication processing using the reference signal RS2 and the detection signal DS to generate a multiplication signal MS2. The low-pass filter 212 (212b) cuts a specific frequency component of the multiplication signal MS2, to thereby generate a processed signal PS2, which is a direct-current component. The signal processing device 214 performs signal processing using the obtained processed signal PS and processed signal PS2, and thereby, the analyte can be detected with higher accuracy.

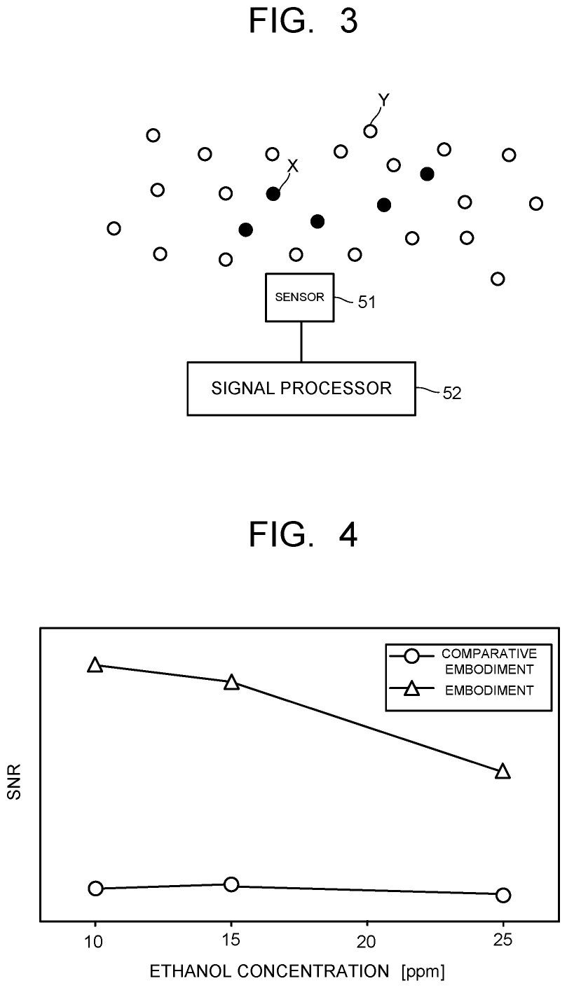

FIG. 3 is a schematic view illustrating an example of a conventional gas detection system. The gas detection system illustrated in FIG. 3 uses a sensor 51 that directly detects an analyte X, in which the sensor 51 detects the analyte X in a state where the analytes X and substances Y that are not to be analyzed are mixed together, to generate a detection signal and a signal processor 52 processes the detection signal to generate a processed signal.

However, the conventional gas detection system using the sensor that directly detects the analyte in the sample gas has difficulty in detecting the analyte because signal components based on the analyte are buried in the noise contained in the detection signal.

Further, known another examples of the conventional gas detection system has a lock-in amplifier and irradiates a sample gas with light to detect the intensity of the light transmitted through the sample gas, to thereby detect the concentration of an analyte. This system utilizes the mechanism in which the amount of light absorbed by the sample gas varies according to the concentration of the analyte. This variation is detected by detecting the intensity of the transmitted light with a photodetector. This light detection is performed by varying the intensity of the light to be emitted to detect the analyte.

However, the example of the conventional gas detection system detects the concentration of the analyte as the amount of light absorbed. Therefore, when detecting a trace amount of analyte floating in the air, the amount of light absorbed by the analyte is minute and the example of the conventional gas detection system has difficulty in detecting this variation.

Thus, the gas detection system in the embodiment uses the gas controller 4, the gas controller 4 varies the concentration of the analyte in the sample gas SG with time, and thereby the waveform of the detection signal DS is controlled. The voltage of the detection signal DS varies periodically as the gas controller 4 varies the concentration of the analyte in the sample gas SG with time. Therefore, the detection signal DS forms a periodic signal containing noise. When the concentration of the analyte is increased, the voltage of the detection signal DS can be increased. When the concentration of the analyte is lowered, the voltage of the detection signal DS can be lowered.

The gas controller 4 can vary the concentration of the analyte in the sample gas SG with time based on the reference signal RS so as to make the detection signal DS synchronize with the reference signal RS. Here, “a plurality of signals synchronize” means that “the amplitudes and phases of the signals match.”

An example of the gas controller 4 includes a measuring object gas supply source 41, a reference gas supply source 42, a valve 43, a valve 44, a valve 45, and a control system 46.

The measuring object gas supply source 41 supplies an analyte gas AG containing an analyte. Examples of the analyte include gaseous moisture (water vapor), volatile organic compounds (such as benzene, toluene, xylene, hexane, pentane, methanol, ethanol, acetone, ethyl acetate, chloroform, dichloromethane, chlorofluorocarbons, formaldehyde, and furfural), geosmin and 2-methylisoborneol, which cause musty odors, as well as rare gases such as xenon and krypton, substances that are gaseous at normal temperature and pressure such as hydrogen, oxygen, nitrogen, nitrogen oxides, hydrogen sulfide, ammonia, carbon monoxide, carbon dioxide, methane, ethane, propane, ethylene, and acetylene, 2,4,6-trinitrotoluene, dinitrotoluene, methamphetamine, amphetamine, and so on. The analyte gas AG may contain a plurality of analytes. The measuring object gas supply source 41 may include a tank that accommodates the analyte gas AG and a pump for supplying the analyte gas AG. The analyte gas AG may be supplied through a filter from outside the measuring object gas supply source 41.

The reference gas supply source 42 supplies a reference gas RG containing no analyte. The reference gas RG contains at least one of oxygen and nitrogen, for example. Examples of the reference gas RG include an oxygen gas, a nitrogen gas, air, and a rare gas. The reference gas supply source 42 may include a tank that accommodates the reference gas RG and a pump for supplying the reference gas RG. The reference gas RG may be supplied from a cylinder outside the reference gas supply source 42 or may be supplied from a gas generator outside the reference gas supply source 42.

The valve 43 adjusts the flow rate of the analyte gas AG from the measuring object gas supply source 41. The valve 43 is provided in the middle of a pipe P11 connected to the measuring object gas supply source 41.

The valve 44 adjusts the flow rate of the reference gas RG from the reference gas supply source 42. The valve 44 is provided in the middle of a pipe P12 connected to the reference gas supply source 42.

The valve 45 adjusts the flow rate of the sample gas SG. The valve 45 can connect the pipe P11 and the pipe P12 to the pipe P2.

The concentration of the analyte in the sample gas SG can be varied by modifying the ratio between the analyte gas AG and the reference gas RG in the sample gas SG. The ratio between the analyte gas AG and the reference gas RG can be modified by adjusting the flow rate of the analyte gas AG with the valve 43, adjusting the flow rate of the reference gas RG with the valve 44, and adjusting the mixing ratio between the analyte gas AG and the reference gas RG with the valve 45.

The control system 46 generates control signals CS intended for controlling the opening and closing of the valve 43, the valve 44, and the valve 45 based on the reference signal RS input from the signal generator 2. The control signals CS are input to the valve 43, the valve 44, and the valve 45.

A control device such as the control system 46 may be configured using hardware using a processor or the like, for example. Each operation may be stored as an operation program in a computer-readable recording medium such as a memory, and each operation may be executed by appropriately reading the operation program stored in the recording medium by the hardware.

As above, the gas detection system in the embodiment can reduce the effect of noise contained in the detection signal and improve the detection sensitivity of the analyte because the gas controller 4 varies the concentration of the analyte with time, thereby enabling signal processing to be performed in synchronization with the detection signal and the reference signal.

FIG. 4 is a view illustrating an example of a comparison result between an SN ratio of the processed signal PS of the gas detection system in the embodiment and an SN ratio of the detection signal of the gas detection system in a comparative embodiment illustrated in FIG. 3. The analyte is ethanol. The horizontal axis in FIG. 4 indicates the concentration of ethanol in the sample gas, and the vertical axis in FIG. 4 indicates the SN ratio (SNR). The circle marks represent the result of the processed signal PS of the gas detection system in the embodiment, and the triangle marks represent the result of the SN ratio of the detection signal of the gas detection system in the actual comparative embodiment. As illustrated in FIG. 4, the processed signal PS of the gas detection system in the embodiment enables a significant reduction in the SN ratio compared to the detection signal of the gas detection system in the comparative embodiment. The SN ratio of the processed signal PS is preferably 500 or less, for example.

### Second Embodiment

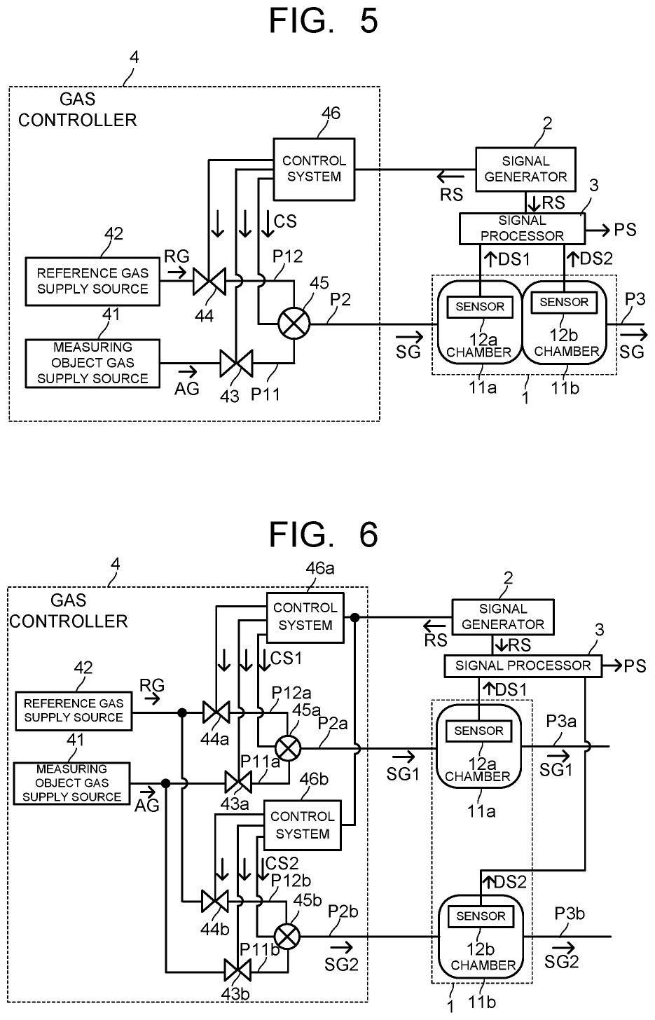

FIG. 5 is a schematic diagram illustrating a configuration example of a gas detection system in a second embodiment. The gas detection system illustrated in FIG. 5 includes the gas detector 1, the signal generator 2, the signal processor 3, and the gas controller 4 as in the first embodiment.

The gas detection system in the second embodiment differs from the gas detection system in the first embodiment in that the gas detector 1 in the second embodiment includes a plurality of sensors 12 (12a, 12b) and a plurality of chambers 11 (11a, 11b).

The chamber 11a allows the sample gas SG to pass therethrough. The chamber 11a is connected to an inlet for supplying the sample gas SG to the chamber 11a and an outlet for discharging the sample gas SG from the chamber 11a. The inlet is connected to the pipe P2. The introduction and discharge of the sample gas SG may be controlled by a pump, which is provided in the middle of the pipe P2 and the pipe P3, for example.

The sensor 12a is provided in the chamber 11a. The sensor 12a is a gas sensor capable of detecting one analyte in the sample gas SG flowing through the chamber 11a. For the other explanation of the sensor 12a, the explanation of the sensor 12 can be used as appropriate.

The chamber 11b allows the sample gas SG supplied from the chamber 11a to pass therethrough. The chamber 11b is connected in series after the chamber 11a. The chamber 11b is connected to an inlet for supplying the sample gas SG to the chamber 11b and an outlet for discharging the sample gas SG from the chamber 11b. The inlet is connected to the inlet of the chamber 11a. The inlet may be connected to the inlet of the chamber 11a via a pipe. The outlet is connected to the pipe P3. The introduction and discharge of the sample gas SG may be controlled by a pump, which is provided in the middle of the pipe P2 and the pipe P3, for example.

The sensor 12b is provided in the chamber 11b. The sensor 12b is a gas sensor capable of detecting one analyte in the sample gas SG flowing through the chamber 11b. The analyte detectable by the sensor 12b may be the same as or different from the analyte detectable by the sensor 12a. For the other explanation of the sensor 12b, the explanation of the sensor 12 can be used as appropriate.

The signal generator 2 includes a signal oscillator capable of generating the reference signal RS corresponding to the analyte. When the detectable analytes are different between the sensors 12a and 12b, the signal generator 2 may include a plurality of signal oscillators to generate a plurality of reference signals RS.

The signal processor 3 can generate the processed signal PS by performing signal processing using a detection signal DS1 and the reference signal RS. The signal processor 3 includes, for example, a signal processing device. The signal processor 3 includes a lock-in amplifier having the configuration illustrated in FIG. 2B, for example. When the detectable analytes are different between the sensors 12a and 12b, a plurality of processed signals PS may be generated by performing signal processing using the detection signal DS1 and the corresponding reference signal RS and performing signal processing using a detection signal DS2 and the corresponding reference signal RS. In this case, switches each may be provided at the connection point between the sensor 12a and the signal processor 3 and the connection point between the sensor 12b and the signal processor 3, and the detection signal DS1 and the detection signal DS2 may be alternately input to a single lock-in amplifier by controlling the switches, or a plurality of lock-in amplifiers may be provided, and the detection signal DS1 and the detection signal DS2 may be separately input to a plurality of the lock-in amplifiers.

The voltage of the detection signal DS1 varies periodically as the gas controller 4 varies the concentration of one analyte in the sample gas SG with time. The voltage of the detection signal DS2 varies periodically as the gas controller 4 varies the concentration of one analyte in the sample gas SG with time.

The gas controller 4 can vary the concentration of one analyte in the sample gas SG with time based on the corresponding reference signal RS so as to make the detection signal DS1 synchronize with the corresponding reference signal RS. The gas controller 4 can vary the concentration of one analyte in the sample gas SG with time based on the corresponding reference signal RS so as to make the detection signal DS2 synchronize with the corresponding reference signal RS. For the other explanation of the gas controller 4, the explanation of the first embodiment can be used as appropriate.

The gas detection system in the second embodiment can improve the dynamic range of gas detection sensitivity and remove noise due to interfering gases by installing a plurality of sensors with different sensitivities or a plurality of sensors for different types of gases. A plurality of sensors may be provided in a single chamber.

This embodiment can be appropriately combined with another embodiment.

### Third Embodiment

FIG. 6 is a schematic diagram illustrating a configuration example of a gas detection system in a third embodiment. The gas detection system illustrated in FIG. 6 includes the gas detector 1, the signal generator 2, the signal processor 3, and the gas controller 4 as in the first embodiment.

The gas detector 1 can detect one or a plurality of analytes in sample gases SG (SG1, SG2) and generate detection signals DS (DS1, DS2). An example of the gas detector 1 includes a plurality of the chambers 11 (11a, 11b) and a plurality of the sensors 12 (12a, 12b), as illustrated in FIG. 6.

The chamber 11a allows the sample gas SG1 to pass therethrough. The chamber 11a is connected to an inlet for supplying the sample gas SG1 to the chamber 11a and an outlet for discharging the sample gas SG1 from the chamber 11a. The inlet is connected to a pipe P2a. The outlet is connected to a pipe P3a. The introduction and discharge of the sample gas SG1 may be controlled by a pump, which is provided in the middle of the pipe P2a and the pipe P3a, for example.

The sensor 12a is provided in the chamber 11a. The sensor 12a is a gas sensor capable of detecting one analyte in the sample gas SG1 flowing through the chamber 11a. For the other explanation of the sensor 12a, the explanation of the sensor 12 can be used as appropriate.

The chamber 11b allows the sample gas SG2 to pass therethrough. The chamber 11b is connected to an inlet for supplying the sample gas SG2 to the chamber 11b and an outlet for discharging the sample gas SG2 from the chamber 11b. The inlet is connected to a pipe P2b. The outlet is connected to a pipe P3b. The introduction and discharge of the sample gas SG2 may be controlled by a pump, which is provided in the middle of the pipe P2b and the pipe P3b, for example.

The sensor 12b is provided in the chamber 11b. The sensor 12b is a gas sensor capable of detecting one analyte in the sample gas SG2 flowing through the chamber 11b. The analyte detectable by the sensor 12b may be the same as or different from the analyte detectable by the sensor 12a. For the other explanation of the sensor 12b, the explanation of the sensor 12 can be used as appropriate.

The signal generator 2 includes a signal oscillator that oscillates the reference signal RS corresponding to the analyte. When the detectable analytes are different between the sensors 12a and 12b, the signal generator 2 may include a plurality of signal oscillators to generate a plurality of reference signals RS.

The signal processor 3 can generate the processed signal PS by performing signal processing using the detection signal DS1 and the reference signal RS. The signal processor 3 includes, for example, a signal processing device. The signal processor 3 includes a lock-in amplifier having the configuration illustrated in FIG. 2B, for example. When the detectable analytes are different between the sensors 12a and 12b, a plurality of processed signals PS may be generated by performing signal processing using the detection signal DS1 and the corresponding reference signal RS and performing signal processing using the detection signal DS2 and the corresponding reference signal RS. In this case, switches each may be provided at the connection point between the sensor 12a and the signal processor 3 and the connection point between the sensor 12b and the signal processor 3, and the detection signal DS1 and the detection signal DS2 may be alternately input to a single lock-in amplifier by controlling the switches, or a plurality of lock-in amplifiers may be provided, and the detection signal DS1 and the detection signal DS2 may be separately input to a plurality of the lock-in amplifiers.

The voltage of the detection signal DS1 varies periodically as the gas controller 4 varies the concentration of one analyte in the sample gas SG1 with time. The voltage of the detection signal DS2 varies periodically as the gas controller 4 varies the concentration of one analyte in the sample gas SG2 with time.

The gas controller 4 can vary the concentration of the analyte in the sample gas SG1 with time based on the corresponding reference signal RS so as to make the detection signal DS1 synchronize with the corresponding reference signal RS. The gas controller 4 can vary the concentration of the analyte in the sample gas SG2 with time based on the corresponding reference signal RS so as to make the detection signal DS2 synchronize with the corresponding reference signal RS.

An example of the gas controller 4 includes the measuring object gas supply source 41, the reference gas supply source 42, valves 43 (43a, 43b), valves 44 (44a, 44b), valves 45 (45a, 45b), and control systems 46 (46a, 46b), as illustrated in FIG. 6. For the other explanations of the measuring object gas supply source 41 and the reference gas supply source 42, the explanation of the first embodiment can be used as appropriate.

The valve 43a adjusts the flow rate of the analyte gas AG from the measuring object gas supply source 41. The valve 43a is provided in the middle of a pipe P11a connected to the measuring object gas supply source 41.

The valve 43b adjusts the flow rate of the analyte gas AG from the measuring object gas supply source 41. The valve 43b is provided in the middle of a pipe P11b connected to the measuring object gas supply source 41. The pipe P11b may be connected to the pipe P11a.

The valve 44a adjusts the flow rate of the reference gas RG from the reference gas supply source 42. The valve 44a is provided in the middle of a pipe P12a connected to the reference gas supply source 42.

The valve 44b adjusts the flow rate of the reference gas RG from the reference gas supply source 42. The valve 44b is provided in the middle of a pipe P12b connected to the reference gas supply source 42. The pipe P12b may be connected to the pipe P12a.

The valve 45a adjusts the flow rate of the sample gas SG1. The valve 45a can connect the pipe P11a and the pipe P12a to the pipe P2a connected to the inlet of the chamber 11a.

The valve 45b adjusts the flow rate of the sample gas SG2. The valve 45b can connect the pipe P11b and the pipe P12b to the pipe P2b connected to the inlet of the chamber 11b.

The concentrations of the analytes in the sample gases SG1, SG2 can be varied by modifying the ratio between the analyte gas AG and the reference gas RG. The ratio between the analyte gas AG and the reference gas RG can be modified by adjusting the flow rate of the analyte gas AG with the valves 43a, 43b, adjusting the flow rate of the reference gas RG with the valves 44a, 44b, and adjusting the mixing ratio between the analyte gas AG and the reference gas RG with the valves 45a, 45b. The ratio between the analyte gas Ag and the reference gas RG in the sample gas SG2 may differ from the ratio between the analyte gas AG and the reference gas RG in the sample gas SG1.

The control system 46a generates control signals CS1 intended for controlling the opening and closing of the valve 43a, the valve 44a, and the valve 45a based on the reference signal RS input from the signal generator 2. The control signals CS1 are input to the valve 43a, the valve 44a, and the valve 45a respectively.

The control system 46b generates control signals CS2 intended for controlling the opening and closing of the valve 43b, the valve 44b, and the valve 45b based on the reference signal RS input from the signal generator 2. The control signals CS2 are input to the valve 43b, the valve 44b, and the valve 45b.

The control systems 46a, 46b may be configured using hardware using a processor or the like, for example. Each operation may be stored as an operation program in a computer-readable recording medium such as a memory, and each operation may be executed by appropriately reading the operation program stored in the recording medium by the hardware.

The gas detection system in the third embodiment includes a plurality of chambers in parallel to the gas flow, and supplies the same or another sample gas to the respective chambers. This can prevent variations in synchronization disturbance of the sample gases among a plurality of the chambers, and thus improve the gas detection sensitivity.

This embodiment can be appropriately combined with another embodiment.

### Fourth Embodiment

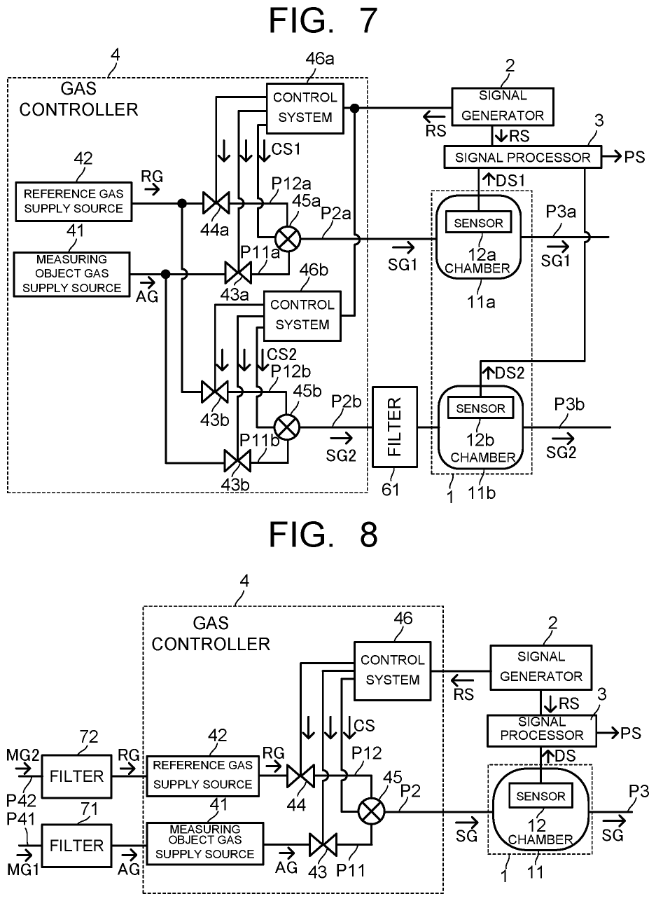

FIG. 7 is a schematic diagram illustrating a configuration example of a gas detection system in a fourth embodiment. The gas detection system illustrated in FIG. 7 includes the gas detector 1, the signal generator 2, the signal processor 3, and the gas controller 4 as in the third embodiment.

The gas detection system in the fourth embodiment differs from the gas detection system in the third embodiment in that it includes a filter 61 in the middle of the connection point between the gas detector 1 and the gas controller 4. For the other explanation, the explanation of the third embodiment can be used as appropriate.

The filter 61 can remove impurities from the sample gas SG2 supplied from the gas controller 4 to the chamber 11b. Examples of the impurities include water vapor, carbon dioxide, oxidizing gases such as hydrogen sulfide, fine particles, and microorganisms. The filter 61 is provided in the middle of the pipe P2b. Examples of the filter 61 include a filter filled with an adsorbent that adsorbs and removes a specific gas, and a membrane filter.

The gas detection system in the fourth embodiment includes the filter 61 at the connection point between the gas controller 4 and the chamber 11b. This, for example, can remove impurities, which are contained in the sample gas SG2 and cause noise when detecting the analyte, and thus improve the detection sensitivity of the analyte.

This embodiment can be appropriately combined with another embodiment.

### Fifth Embodiment

FIG. 8 is a schematic diagram illustrating a configuration example of a gas detection system in a fifth embodiment. The gas detection system illustrated in FIG. 8 includes the gas detector 1, the signal generator 2, the signal processor 3, and the gas controller 4 as in the fifth embodiment.

The gas detection system in the fifth embodiment differs from the gas detection system in the first embodiment in that it includes a filter 71 in front of the measuring object gas supply source 41 and a filter 72 in front of the reference gas supply source 42. For the other explanation, the explanation of the first embodiment can be used as appropriate.

The filter 71 removes a first impurity from a mixed gas MG1, and thereby the analyte gas AG is produced. Examples of the first impurity include water vapor, carbon dioxide, oxidizing gases such as hydrogen sulfide, fine particles, and microorganisms. The filter 71 is provided in the middle of a pipe P41 connected to a gas intake of the measuring object gas supply source 41, for example. Examples of the filter 71 include a filter filled with an adsorbent that adsorbs and removes a specific gas, and a membrane filter.

The filter 72 removes a second impurity from a mixed gas MG2, and thereby the reference gas RG is produced. Examples of the second impurity include water vapor, carbon dioxide, oxidizing gases such as hydrogen sulfide, fine particles, and microorganisms, in addition to the analyte gas AG. The filter 72 is provided in the middle of a pipe P42 connected to a gas intake of the reference gas supply source 42, for example. Examples of the filter 72 include a filter filled with an adsorbent that adsorbs and removes a specific gas, and a membrane filter.

The gas detection system in the fifth embodiment includes the filter 71 disposed so as to precede the measuring object gas supply source 41 and includes the filter 72 disposed so as to precede the reference gas supply source 42, and the filter 71 and the filter 72 remove impurities that cause noise when detecting the analyte. This can improve the detection sensitivity of the analyte.

This embodiment can be appropriately combined with another embodiment.

It should be noted that the structures of the embodiments may be employed in combination, or part thereof may be modified. While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The embodiments can be summarized into the following clauses.

### (Clause 1)

A gas detection system, comprising:
a gas detector that detects a first analyte in a first sample gas to generate a first detection signal;
a signal generator that generates a first reference signal corresponding to the first analyte;
a signal processor that performs first signal processing using the first detection signal and the first reference signal, to thereby generate a first processed signal; and
a gas controller that varies the concentration of the first analyte in the first sample gas with time based on the first reference signal so as to make the first detection signal synchronize with the first reference signal.

### (Clause 2)

The gas detection system according to the clause 1, wherein the gas detector includes:
a first chamber through which the first sample gas flows; and
a first sensor provided in the first chamber, the first sensor being configured to detect the first analyte in the first sample gas flowing through the first chamber to generate the first detection signal.

### (Clause 3)

The gas detection system according to the clause 1 or the clause 2, wherein the gas controller includes:
a first gas supply source that supplies a measuring object gas containing the first analyte;
a second gas supply source that supplies a reference gas that does not contain the first analyte;
a first valve that adjusts the flow rate of the measuring object gas from the first gas supply source;
a second valve that adjusts the flow rate of the reference gas from the second gas supply source; and
a control system that generates a first control signal that controls opening and closing of the first valve and generates a second control signal that controls opening and closing of the second valve according to the first reference signal, and
the gas controller varies the concentration of the first analyte in the first sample gas with time by controlling opening and closing of the first valve and opening and closing of the second valve according to the first control signal and the second control signal to modify the ratio between the measuring object gas and the reference gas in the first sample gas.

### (Clause 4)

The gas detection system according to the clause 1, wherein
the gas detector includes:
a first chamber through which the first sample gas flows;
a first sensor provided in the first chamber, the first sensor being configured to detect the first analyte in the first sample gas flowing through the first chamber to generate the first detection signal;
a second chamber connected in series with the first chamber, the second chamber through which the first sample gas flows; and
a second sensor provided in the second chamber, the second sensor being configured to detect a second analyte in the first sample gas flowing through the second chamber to generate a second detection signal,
the signal generator generates a second reference signal corresponding to the second analyte,
the signal processor performs second signal processing using the second detection signal and the second reference signal, to thereby generate a second processed signal, and
the gas controller varies the concentration of the second analyte in the first sample gas with time based on the second reference signal so as to make the second detection signal synchronize with the second reference signal.

### (Clause 5)

The gas detection system according to the clause 1, wherein
the gas detector includes:
a first chamber through which the first sample gas flows;
a first sensor provided in the first chamber, the first sensor being configured to detect the first analyte in the first sample gas flowing through the first chamber to generate a first detection signal;
a second chamber through which a second sample gas flows; and
a second sensor provided in the second chamber, the second sensor being configured to detect a second analyte in the second sample gas flowing through the second chamber to generate a second detection signal,
the signal generator generates a second reference signal corresponding to the second analyte,
the signal processor performs second signal processing using the second detection signal and the second reference signal, to thereby generate a second processed signal,
the gas controller includes:
a first gas supply source that supplies a measuring object gas containing the first analyte and the second analyte;
a second gas supply source that supplies a reference gas that does not contain the first analyte or the second analyte;
a first valve that adjusts the flow rate of the measuring object gas from the first gas supply source;
a second valve that adjusts the flow rate of the reference gas from the second gas supply source;
a first control system that generates a first control signal that controls opening and closing of the first valve and generates a second control signal that controls opening and closing of the second valve according to the first reference signal;
a third valve that adjusts the flow rate of the measuring object gas from the first gas supply source;
a fourth valve that adjusts the flow rate of the reference gas from the second gas supply source; and
a second control system that generates a third control signal that controls opening and closing of the third valve and generates a fourth control signal that controls opening and closing of the fourth valve according to the second reference signal,
the gas controller varies the concentration of the first analyte in the first sample gas with time by controlling the first valve and the second valve according to the first control signal and the second control signal to modify a first ratio between the measuring object gas and the reference gas in the first sample gas, and
the gas controller varies the concentration of the second analyte in the second sample gas with time by controlling the third valve and the fourth valve according to the third control signal and the fourth control signal to modify a second ratio between the measuring object gas and the reference gas in the second sample gas.

### (Clause 6)

The gas detection system according to the clause 1, wherein
the gas detector includes:
a first chamber through which the first sample gas flows;
a first sensor provided in the first chamber, the first sensor being configured to detect the first analyte in the first sample gas flowing through the first chamber to generate the first detection signal;
a second chamber through which a second sample gas flows; and
a second sensor provided in the second chamber, the second sensor being configured to detect the first analyte in the second sample gas flowing through the second chamber to generate a second detection signal,
the signal generator generates a second reference signal corresponding to the first analyte,
the signal processor performs second signal processing using the second detection signal and the second reference signal, to thereby generate a second processed signal,
the gas controller includes:
a first gas supply source that supplies a measuring object gas containing the first analyte;
a second gas supply source that supplies a reference gas that does not contain the first analyte;
a first valve that adjusts the flow rate of the measuring object gas from the first gas supply source;
a second valve that adjusts the flow rate of the reference gas from the second gas supply source;
a first control system that generates a first control signal that controls opening and closing of the first valve and generates a second control signal that controls opening and closing of the second valve according to the first reference signal;
a third valve that adjusts the flow rate of the measuring object gas from the first gas supply source;
a fourth valve that adjusts the flow rate of the reference gas from the second gas supply source; and
a second control system that generates a third control signal that controls opening and closing of the third valve and generates a fourth control signal that controls opening and closing of the fourth valve according to the second reference signal,
the gas controller varies the concentration of the first analyte in the first sample gas with time by controlling the first valve and the second valve according to the first control signal and the second control signal to modify a first ratio between the measuring object gas and the reference gas in the first sample gas, and
the gas controller varies the concentration of the first analyte in the second sample gas with time by controlling the third valve and the fourth valve according to the third control signal and the fourth control signal to modify a second ratio between the measuring object gas and the reference gas in the second sample gas.

### (Clause 7)

The gas detection system according to the clause 6, further comprising:
a filter that removes an impurity from the second sample gas supplied to the second chamber from the gas controller.

### (Clause 8)

The gas detection system according to any one of the clause 1 to the clause 3, wherein
the signal processor includes a lock-in amplifier that generates the first processed signal by performing the first signal processing using the first detection signal and the first reference signal that are synchronized.

### (Clause 9)

The gas detection system according to the clause 4 or the clause 5, wherein
the signal processor includes a lock-in amplifier that generates the first processed signal by performing the first signal processing using the synchronized first detection signal and first reference signal and generates the second processed signal by performing the second signal processing using the synchronized second detection signal and second reference signal.

### (Clause 10)

The gas detection system according to the clause 3, the clause 5, or the clause 6, further comprising:
a first filter provided in front of the first gas supply source, the first filter that removes a first impurity from a first mixed gas, to thereby generate the measuring object gas; and
a second filter provided in front of the second gas supply source, the second filter that removes a second impurity from a second mixed gas, to thereby generate the reference gas.

### (Clause 11)

The gas detection system according to any one of the clause 1 to the clause 10, wherein
the first analyte is a volatile organic compound.

### (Clause 12)

The gas detection system according to the clause 4 or the clause 5, wherein the second analyte is different from the first analyte.

### (Clause 13)

A gas detection method, comprising:
a step of detecting a first analyte in a first sample gas to generate a first detection signal;
a step of generating a first reference signal corresponding to the first analyte;
a step of performing first signal processing using the first detection signal and the first reference signal, to thereby generate a first processed signal; and
a step of varying the concentration of the first analyte in the first sample gas based on the first reference signal so as to make the first detection signal synchronize with the first reference signal.

What is claimed is:

1. A gas detection system, comprising:
a gas detector that detects a first analyte in a first sample gas to generate a first detection signal;
a signal generator that generates a first reference signal corresponding to the first analyte;
a signal processor that performs first signal processing using the first detection signal and the first reference signal, to thereby generate a first processed signal; and
a gas controller that varies the concentration of the first analyte in the first sample gas with time based on the first reference signal so as to make the first detection signal synchronize with the first reference signal.

2. The gas detection system according to claim 1, wherein
the gas detector includes:
a first chamber through which the first sample gas flows; and
a first sensor provided in the first chamber, the first sensor being configured to detect the first analyte in the first sample gas flowing through the first chamber to generate the first detection signal.

3. The gas detection system according to claim 1, wherein
the gas controller includes:
a first gas supply source that supplies a measuring object gas containing the first analyte;
a second gas supply source that supplies a reference gas that does not contain the first analyte;
a first valve that adjusts the flow rate of the measuring object gas from the first gas supply source;
a second valve that adjusts the flow rate of the reference gas from the second gas supply source; and
a control system that generates a first control signal that controls opening and closing of the first valve and generates a second control signal that controls opening and closing of the second valve according to the first reference signal, and
the gas controller is configured to vary the concentration of the first analyte in the first sample gas with time by controlling opening and closing of the first valve and opening and closing of the second valve according to the first control signal and the second control signal to modify the ratio between the measuring object gas and the reference gas in the first sample gas.

4. The gas detection system according to claim 1, wherein
the gas detector includes:
a first chamber through which the first sample gas flows;
a first sensor provided in the first chamber, the first sensor being configured to detect the first analyte in the first sample gas flowing through the first chamber to generate the first detection signal;
a second chamber connected in series with the first chamber, the second chamber through which the first sample gas flows; and
a second sensor provided in the second chamber, the second sensor being configured to detect a second analyte in the first sample gas flowing through the second chamber to generate a second detection signal,
the signal generator is configured to generate a second reference signal corresponding to the second analyte,
the signal processor performs second signal processing using the second detection signal and the second reference signal, to thereby generate a second processed signal, and
the gas controller is configured to vary the concentration of the second analyte in the first sample gas with time based on the second reference signal so as to make the second detection signal synchronize with the second reference signal.

5. The gas detection system according to claim 1, wherein
the gas detector includes:
a first chamber through which the first sample gas flows;
a first sensor provided in the first chamber, the first sensor being configured to detect the first analyte in the first sample gas flowing through the first chamber to generate a first detection signal;
a second chamber through which a second sample gas flows; and
a second sensor provided in the second chamber, the second sensor being configured to detect a second analyte in the second sample gas flowing through the second chamber to generate a second detection signal,
the signal generator generates a second reference signal corresponding to the second analyte,
the signal processor performs second signal processing using the second detection signal and the second reference signal, to thereby generate a second processed signal,
the gas controller includes:
a first gas supply source that supplies a measuring object gas containing the first analyte and the second analyte;
a second gas supply source that supplies a reference gas that does not contain the first analyte or the second analyte;
a first valve that adjusts the flow rate of the measuring object gas from the first gas supply source;
a second valve that adjusts the flow rate of the reference gas from the second gas supply source;
a first control system that generates a first control signal that controls opening and closing of the first valve and generates a second control signal that controls opening and closing of the second valve according to the first reference signal;
a third valve that adjusts the flow rate of the measuring object gas from the first gas supply source;
a fourth valve that adjusts the flow rate of the reference gas from the second gas supply source; and
a second control system that generates a third control signal that controls opening and closing of the third valve and generates a fourth control signal that controls opening and closing of the fourth valve according to the second reference signal,
the gas controller varies the concentration of the first analyte in the first sample gas with time by controlling the first valve and the second valve according to the first control signal and the second control signal to modify a first ratio between the measuring object gas and the reference gas in the first sample gas, and
the gas controller varies the concentration of the second analyte in the second sample gas with time by controlling the third valve and the fourth valve according to the third control signal and the fourth control signal to modify a second ratio between the measuring object gas and the reference gas in the second sample gas.

6. The gas detection system according to claim 1, wherein
the gas detector includes:
a first chamber through which the first sample gas flows;
a first sensor provided in the first chamber, the first sensor being configured to detect the first analyte in the first sample gas flowing through the first chamber to generate the first detection signal;
a second chamber through which a second sample gas flows; and
a second sensor provided in the second chamber, the second sensor being configured to detect the first analyte in the second sample gas flowing through the second chamber to generate a second detection signal,
the signal generator generates a second reference signal corresponding to the first analyte,
the signal processor performs second signal processing using the second detection signal and the second reference signal, to thereby generate a second processed signal,
the gas controller includes:
a first gas supply source that supplies a measuring object gas containing the first analyte;
a second gas supply source that supplies a reference gas that does not contain the first analyte;
a first valve that adjusts the flow rate of the measuring object gas from the first gas supply source;
a second valve that adjusts the flow rate of the reference gas from the second gas supply source;
a first control system that generates a first control signal that controls opening and closing of the first valve and generates a second control signal that controls opening and closing of the second valve according to the first reference signal;
a third valve that adjusts the flow rate of the measuring object gas from the first gas supply source;
a fourth valve that adjusts the flow rate of the reference gas from the second gas supply source; and
a second control system that generates a third control signal that controls opening and closing of the third valve and generates a fourth control signal that controls opening and closing of the fourth valve according to the second reference signal,
the gas controller varies the concentration of the first analyte in the first sample gas with time by controlling the first valve and the second valve according to the first control signal and the second control signal to modify a first ratio between the measuring object gas and the reference gas in the first sample gas, and
the gas controller varies the concentration of the first analyte in the second sample gas with time by controlling the third valve and the fourth valve according to the third control signal and the fourth control signal to modify a second ratio between the measuring object gas and the reference gas in the second sample gas.

7. The gas detection system according to claim 6, further comprising:
a filter that removes an impurity from the second sample gas supplied to the second chamber from the gas controller.

8. The gas detection system according to claim 1, wherein
the signal processor includes a lock-in amplifier that generates the first processed signal by performing the first signal processing using the first detection signal and the first reference signal that are synchronized.

9. The gas detection system according to claim 4, wherein
the signal processor includes a lock-in amplifier that generates the first processed signal by performing the first signal processing using the synchronized first detection signal and first reference signal and generates the second processed signal by performing the second signal processing using the synchronized second detection signal and second reference signal.

10. The gas detection system according to claim 3, further comprising:
a first filter provided in front of the first gas supply source, the first filter that removes a first impurity from a first mixed gas, to thereby generate the measuring object gas; and
a second filter provided in front of the second gas supply source, the second filter that removes a second impurity from a second mixed gas, to thereby generate the reference gas.

11. The gas detection system according to claim 1, wherein
the first analyte is a volatile organic compound.

12. The gas detection system according to claim 4, wherein
the second analyte is different from the first analyte.

13. A gas detection method, comprising:
detecting a first analyte in a first sample gas to generate a first detection signal;
generating a first reference signal corresponding to the first analyte;
performing first signal processing using the first detection signal and the first reference signal, to thereby generate a first processed signal; and
varying the concentration of the first analyte in the first sample gas based on the first reference signal to so as make the first detection signal synchronize with the first reference signal.
